(19) United States
(12) Patent Application Publication
(10) Pub. No.: US 2002/0177831 A1
(43) Pub. Date: Nov. 28, 2002

(54) Personal care absorbent with inherent transfer delay

(75) Inventors: Michael Allen Daley, Alpharetta, GA (US); Tamara Lee Mace, Doraville, GA (US); David Michael Matela, Alpharetta, GA (US)

Correspondence Address: James B. Robinson, Kimberly-Clark Worldwide, Inc., Patent Department, 401 North Lake Street, Neenah, WI, 54956, US

(73) Assignee: Kimberly-Clark Worldwide, Inc.

(21) Appl. No.: 09/865,836

(22) Filed: May 25, 2001

(51) Int. Cl. A61F 13/15

(52) U.S. Cl. 604/385.01

(57) ABSTRACT

There is provided a personal care absorbent article having an absorbent core that has been treated in a manner, or is made from materials, that inhibit the transfer of liquid through the structure in the target area. The distribution layer above the core could likewise be treated in a manner that discourages Z-directional fluid movement. A separate transfer delay layer is avoided, thereby simplifying manufacture and reducing costs.

A number of transfer delay treatments are possible. These include increasing the density of the upper layer of the absorbent core or lower layer of the distribution layer below the target area, making the absorbent core below the target area of rich in superabsorbent, making all or part of the core below the target area from very slow absorbing superabsorbents, including a soluble binder in the core below the target area, treating the core or distribution layer below the target area with a hydrophobic treatment, or combinations of these methods.

### BACKGROUND OF THE INVENTION

This invention concerns absorbent articles that are useful in personal care products like disposable sanitary napkins, diapers, training pants, incontinence garments, wound care products and the like. These articles typically include a body side liner, a liquid impervious outer layer or “baffle”, and an absorbent core between the liner and the baffle, More particularly, this invention relates to absorbent systems that must manage complex viscous body fluids such as menses and wound exudates.

Personal care products are intended to intake and retain body fluids. Desired performance objectives of these articles include low leakage from the product and a dry feeling for the wearer. Currently available products suffer from higher than desired leakage levels that may produce stains on bedding and clothing. As a result, they are not perceived by users to fully deliver on other consumer desires such as dryness, fit, comfort and fluid retention. Leakage can occur due to a variety of shortcomings in the product, one of which is an insufficient rate of fluid intake by the absorbent system, particularly on the second or third liquid surges. This is a particular problem for feminine care products intended for overnight use where significant fluid retention capacity is required in order to hold the majority of the fluid. Most commercially available sanitary pads, for example, have relatively high leakage rates, failing as much as 30% of the time. This is believed to be due to the highly viscous nature of menses and the great variability in delivery volume that results in overloading of the pad in the target area and subsequent leaking. Insufficient distribution of menses is believed to be one of the key causes of the target area overloading.

Non-woven materials such as carded webs and spunbond webs have been used as the body side liner in absorbent products. Open, porous liner structures have been employed to allow liquid to pass through them rapidly and help keep the wearer's skin separated from the wet absorbent pad beneath the liner. Some structures have incorporated zoned surfactant treatments in selected areas of the liners to increase the wettability of the selected regions and thereby control the amount of liquid wet-back onto a wearer's skin. In addition, other layers of material, such as those constructed with thick, lofty fabric structures, have been interposed between the liner and absorbent pad for the purpose of reducing wet-back.

The outer cover or baffle is designed to be impermeable to liquid in order to keep the clothing or bedding of the wearer from becoming soiled. The impermeable baffle is preferably made from a thin film and is generally made from plastic, though other materials may be used. Nonwoven webs, films or film coated nonwovens may be used as the baffle as well. Suitable film compositions for the baffle include polyethylene film which may have an initial thickness of from about 0.5 mil (0.012 millimeter) to about 5.0 mil (0.12 millimeter). The baffle may optionally be composed of a vapor or gas permeable, microporous “breathable” material, that is permeable to vapors or gas yet substantially impermeable to liquid.

Absorbent articles have employed various types of absorbent cores composed of cellulosic fibers. Particular absorbent garments may be configured to control the distribution of absorbed liquids. An absorbent article, for example, can have a liquid permeable distribution layer which is located between a top sheet layer and an absorbent body. In other configurations, a conventional absorbent article can have fluid storage and acquisition zones composed of cellulosic fluff mixed with absorbent gelling particles and may include a multi-layer absorbent core arrangement having varying compositions.

Conventional fluff-based absorbent structures, such as those discussed above, have cellulosic fibers which when wetted can lose resiliency and collapse. As a result, the liquid uptake rate of the wetted structures may become too low to adequately accommodate subsequent, successive liquid surges. In articles, in which absorbent gelling particles are included between the fibers to hold them apart, the gelling particles swell and do not release the absorbed fluid. Swelling of the particles can diminish the void volume of the absorbent structure and reduce the ability of the structure to uptake fluids.

Distribution of liquid in the field of urine management in products like diapers is often provided by materials that have small pores with a narrow pore size distribution. These distribution materials move the high volume, low viscosity urine insults out of the target area, and ideally do so in enough time for the target area to be able to accept the next insult. The movement of urine may be to relatively remote parts of the diaper, overcoming substantial hydrostatic pressure. Feminine hygiene products experience lower total insult volumes, but the fluid is of greater viscosity, making it more difficult to move. Distribution materials must be quite different for feminine hygiene products than for products concerned primarily with urine management.

An additional layer may be present in absorbent structures and acts to slow the downward (Z-directional) movement of fluid and encourage the fluid to move in the X-Y (lateral) plane. This layer is known as a “transfer delay” layer and may be, for example, a perforated film, meltblown fabric or other single or multi-layer material. By encouraging the spreading of fluid before absorption into the core, the transfer delay layer uses more of the core. This allows the core to be thinner in the target area and is a more efficient and cost effective use of materials than found in embodiments without such a layer.

Despite the development of absorbent structures as discussed above, there remains a need for improved absorbent structures that can adequately reduce leakage from absorbent products, such as feminine hygiene products and be simpler to manufacture and still more cost effective. There is a need for an absorbent structure that can provide improved handling of liquid surges by more effectively intaking, distributing and retaining repeated loadings of liquid.

### SUMMARY OF THE INVENTION

In response to the discussed difficulties and problems encountered in the prior art, a new structural composite comprising a feminine hygiene product having good distribution and transfer performance to allow movement of menses from a target area is provided.

This is achieved by a personal care absorbent article having an absorbent core or distribution layer that has been treated in a manner, or is made from materials, that inhibit the transfer of liquid through the structure in the area below the target area. A separate transfer delay layer is avoided, thereby simplifying manufacture and reducing costs. In this invention, fluid moves in the Z-direction at a slower rate in the area of the target area and below, than it does outside the target area.

A number of transfer delay treatments are possible. These include increasing the density of the upper layer of the absorbent core below the target area, making the absorbent core below the target area rich in superabsorbent, particularly superabsorbent fibers (SAF), making all or part of the absorbent core below the target area from very slow absorbing superabsorbents, including a soluble binder in the absorbent core below the target area, treating the absorbent core below the target area with a hydrophobic treatment, or combinations of these methods. These methods may also be applied to the layer immediately above the core and will be similarly effective.

A feminine hygiene product incorporating this invention would preferably have an outer baffle, a liner and an absorbent fluff core and the fluid would move more slowly in the Z-direction in the target area than outside of it.

### DEFINITIONS

As used herein, the following terms have the definitions ascribed to them.

The term “disposable” includes being disposed of after use and not intended to be washed and reused.

As used herein, the term “nonwoven fabric or web” means a web having a structure of individual fibers or threads which are interlaid, but not in an identifiable manner, as in a knitted fabric. Nonwoven fabrics or webs have been formed from many processes such as, for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

As used herein, the term “spunbonded fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., U.S. Pat. No. 3,692,618 to Dorschner et al., U.S. Pat. No. 3,802,817 to Matsuki et al., U.S. Pat. No. 3,338,992 and U.S. Pat. No. 3,341,394 to Kinney, U.S. Pat. No. 3,502,763 to Hartmann, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns. The fibers may also have shapes such as those described in U.S. Pat. No. 5,277,976 to Hogle et al., U.S. Pat. No. 5,466,410 to Hills, and U.S. Pat. No. 5,069,970 and U.S. Pat. No. 5,057,368 to Largman et al., which describe hybrids with unconventional shapes.

As used herein, the term “meltblown fibers” means fibers formed by extruding a molten thermoplastic material through a plurality of fine, usually circular, die capillaries as molten threads or filaments into converging high velocity, usually hot, gas (for example, air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than 10 microns in average diameter, and are generally tacky when deposited onto a collecting surface.

As used herein, the term “bonded carded web” refers to webs made from staple fibers which are sent through a combing or carding unit, which breaks apart and aligns the staple fibers in the machine direction to form a generally machine direction-oriented fibrous nonwoven web. Such fibers are usually purchased in bales which are placed in a picker which separates the fibers prior to the carding unit. Once the web is formed, it is then bonded by one or more of several known bonding methods. One such bonding method is powder bonding, wherein a powdered adhesive is distributed through the web and then activated, usually by heating the web and adhesive with hot air. Another suitable bonding method is pattern bonding, wherein heated calender rolls or ultrasonic bonding equipment are used to bond the fibers together, usually in a localized bond pattern, though the web can be bonded across its entire surface, if so desired. Another suitable and well-known bonding method, particularly when using bicomponent staple fibers, is through-air bonding.

“Airlaying” is a well known process by which a fibrous nonwoven layer can be formed. In the airlaying process, bundles of small fibers having typical lengths ranging from about 6 to about 52 millimeters (mm) are separated and entrained in an air supply and then deposited onto a forming screen, usually with the assistance of a vacuum supply. The randomly deposited fibers then are bonded to one another using, for example, hot air or a spray adhesive. Airlaying is taught in, for example, U.S. Pat. No. 4,640,810 to Laursen et al. and U.S. Pat. No. 5,885,516 to Mosgaard.

“Hydrophilic” describes fibers or the surfaces of fibers which are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials can be provided by a Cahn SFA-222 Surface Force Analyzer System, or a substantially equivalent system. When measured with this system, fibers having contact angles less than 90° are designated “wettable” or “hydrophilic”, while fibers having contact angles equal to or greater than to 90° are designated “nonwettable” or hydrophobic.

As used herein, the term “personal care product” or “personal care absorbent product” means diapers, training pants, absorbent underpants, adult incontinence products, bandages and other wound care products and feminine hygiene products.

“Target area” refers to the surface area or position on a personal care product where an insult is normally delivered by a wearer. The size, shape and location of this area will, of course, vary, depending on the type and size of product involved. Feminine hygiene products, for example, have a generally oblong target area about 2 inch (5.1 cm) long by 4 inch (10.2 cm) wide at their longest dimensions. A diaper may have a target area area about 3.5 inch (8.8 cm) long by 8 inch (20.4 cm) wide and an incontinence product for adults may have a target area still larger. The target area for a bandage may cover a larger percentage of the surface than would a target area for a diaper.

### TEST METHODS

### Absorption Time Index (ATI)

In this test the absorbent capacity of a superabsorbent material is determined versus time for up to 200 minutes under light pressure, e.g. about 0.01 psi.

A one inch (25.4 mm) inside diameter cylinder with an integral 100 mesh stainless steel screen on one end is used to hold 0.16±0.005 grams of dry superabsorbent. The superabsorbent should be carefully placed in the cylinder so that superabsorbent does not stick to the sides of the cylinder. The cylinder should be tapped gently to more evenly distribute the superabsorbent on the screen. A 4.4 gram, 0.995 inch (252.73 mm) diameter plastic piston is then placed in the cylinder and the cylinder, piston and superabsorbent assembly weighed. The assembly is placed in a 3 inch by 3 inch (76.4 mm by 76.4 mm) liquid basin having a 0.875 weight percent NaCl saline solution to a depth of 1 cm. Tap the cylinder gently to remove any air trapped under it and maintain the saline solution depth at 1 cm throughout the test.

Use a timer capable of reading 200 minutes in one second intervals. Start the timer and after 5 minutes in the solution, remove the assembly and blot on absorbent paper. A preferred paper is Kleenex® Premium Dinner Napkins from Kimberly-Clark Corp. though any other effective paper may be used. In blotting, press the paper tightly against the cylinder to ensure good contact. Touch the cylinder three times to dry paper and there should be very little liquid removed the third time. Weigh the assembly and return assembly to the liquid basin. Blotting and weighing should take about 5 seconds and the timer should be kept running throughout the test. Take readings at 5, 10, 15, 30, 45, 60, 75, 90, 120, 160 and 200 minutes. Use fresh dry napkins for each reading.

After the final reading, calculate the grams of liquid absorbed per gram of superabsorbent. The amount of liquid absorbed at particular times divided by the amount absorbed at 200 minutes may be plotted versus time for a graphical representation of the absorption rate.

The ATI is calculated as follows:

ATI=(t₁₀+t₂₀+t₃₀+t₄₀+t₅₀+t₆₀+t₇₀+t₈₀+t₉₀)/9

where tₙ is the time in minutes at which n percentage of the absorbent capacity at 200 minutes is used, e.g. t₃₀ is the time at which 30 percent of the total capacity is used.

### DETAILED DESCRIPTION

This invention relates to personal care absorbent articles such as disposable sanitary napkins, diapers, incontinence garments, and the like. These products typically have a liquid permeable body side liner, a liquid impervious baffle, and an absorbent core between the liner and baffle.

The liner is designed to be highly permeable to liquid and to be non-irritating to the skin. Such a liner allows urine and menses to penetrate through itself quite easily and feels soft to the skin. The liner may be made from various materials including nonwoven webs, apertured films, foams and combinations thereof. The nonwovens and films may be made from synthetic polymers similar to the baffle, including polyolefins like polyethylene and polypropylene. The nonwovens may also be made from natural fibers or combinations of natural and synthetic fibers. Liners may also be made from creped materials such as creped nonwoven webs.

The liner may optionally have more than one layer or may have one layer in a central area with multiple layers in the side areas. The opposite configuration is also possible with two or more layers in the central area and only one on the sides. Such a liner may be advantageous for menstrual use or for delivery of medicaments.

More sophisticated types of liners may incorporate treatments of lotions or medicaments to improve the environment near the skin or to actually improve skin health. Such treatments include aloe, vitamin E, baking soda and other preparations as may be known or developed by those skilled in the art.

The outer cover or “baffle” is designed to be impermeable to liquid in order to keep the clothing or bedding of the wearer from becoming soiled. The impermeable baffle is preferably made from a thin film and is generally made from plastic though other materials may be used. Nonwoven webs, films or film coated nonwovens may be used as the baffle as well. Suitable film compositions for the baffle include polyethylene film which may have an initial thickness of from about 0.5 mil (0.012 millimeter) to about 5.0 mil (0.12 millimeter). The baffle may optionally be composed of a vapor or gas permeable, microporous “breathable” material, that is permeable to vapors or gas yet substantially impermeable to liquid. Breathability can be imparted in polymer films by, for example, using fillers in the film polymer formulation, extruding the filler/polymer formulation into a film and then stretching the film sufficiently to create voids around the filler particles, thereby making the film breathable. Generally, the more filler used and the higher the degree of stretching, the greater the degree of breathability. Other suitable thermoplastic materials like other olefins, nylons, polyesters or copolymers of, for example, polyethylene and polypropylene may also be used.

The core portion of a personal care product is designed to absorb liquids and secondarily to contain solids. The core, known also as a retention layer, may be made with pulp and/or superabsorbent materials. These materials absorb liquids quite quickly and efficiently in order to minimize leakage. Core materials may be made according to a number of processes including the coform process, airlaying, and bonding and carding and should be between 50 and 500 gsm.

Distribution layers also are included in many personal care products. Distribution layers are usually located next to the core and accept liquid from the surge or liner layer and distribute it to other areas of the core using capillary action. Optional transfer delay layers are also located between the distribution layer and core and act to distribute liquid in a more passive manner than distribution layers, i.e., by blocking the Z-directional pathways to the core, detouring fluids into the X-Y plane. One way in which liquid may be delayed or distributed uses a liquid responsive film layer (e.g., PVOH film), partially wrapped with a fluid retention material (fluff/SAM). A surge material is added to the circumference of the film only in the area not wrapped with retention material. The film will resist fluid penetration until the liquid responsive film becomes soluble and so acts to distribute fluid along its length. In these manners, rather than absorbing liquid exclusively in the vicinity of the target area, more of the absorbent core is used.

As mentioned above, the materials of this invention may be made from synthetic polymers, natural fibers, pulps and superabsorbents or combinations thereof. Synthetic fibers include those made from polyolefins, polyamides, polyesters, rayon, acrylics, superabsorbents, LYOCELL® regenerated cellulose and any other suitable synthetic fibers known to those skilled in the art. Many polyolefins are available for fiber production, for example polyethylenes such as Dow Chemical's ASPUN® 681 1A liner low density polyethylene, 2553 LLDPE and 25355 and 12350 high density polyethylene are such suitable polymers. The polyethylenes have melt indices, respectively, of about 26, 40, 25 and 12. Fiber forming polypropylenes include Exxon Chemical Company's ESCORENE® PD 3445 polypropylene and Montell Chemical Co.'s PF304. Other polyolefins are also available.

Natural fibers include wool, cotton, flax, hemp and wood pulp. Wood pulps include standard softwood fluffing grade such as CR-1654 (U.S. Alliance Pulp Mills, Coosa, Ala.) Pulp may be modified in order to enhance the inherent characteristics of the fibers and their processability. Curl may be imparted to the fibers by methods including chemical treatment or mechanical twisting. Curl is typically imparted before crosslinking or stiffening. Pulps may be stiffened by the use of crosslinking agents such as formaldehyde or its derivatives, glutaraldehyde, epichlorohydrin, methylated compounds such as urea or urea derivatives, dialdehydes such as maleic anhydride, non-methylated urea derivatives, citric acid or other polycarboxylic acids. Some of these agents are less preferable than others due to environmental and health concerns. Pulp may also be stiffened by the use of heat or caustic treatments such as mercerization. Examples of these types of fibers include NHB416 which is a chemically crosslinked southern softwood pulp fibers which enhances wet modulus, available from the Weyerhaeuser Corporation of Tacoma, WA. Other useful pulps are debonded pulp (NF405) and non-debonded pulp (NB416) also from Weyerhaeuser. HPZ3from Buckeye Technologies, Inc of Memphis, TN, has a chemical treatment that sets in a curl and twist, in addition to imparting added dry and wet stiffness and resilience to the fiber. Another suitable pulp is Buckeye HP2 pulp and still another is IP Supersoft from International Paper Corporation. Suitable rayon fibers are 1.5 denier Merge 18453 fibers from Acordis Cellulose Fibers Incorporated of Axis, Alabama.

Superabsorbents that may be useful in the present inventions can be chosen from classes based on chemical structure as well as physical form. Superabsorbents may be based on chemistries that include but are not limited to acrylic acid, iso-butylene/maleic anhydride, polyethylene oxide, carboxy-methyl cellulose, poly vinyl pyrrollidone, and poly vinyl alcohol. The superabsorbents may range in rate from slow to fast. The superabsorbents may be in the form of foams, macroporous or microporous particles, fibers, sheets or films, and may have fuzzy or fibrous coatings or morphology. An exemplary superabsorbent may be obtained from Stockhausen, Inc and is designated as FAVOR(® 880. Other examples of superabsorbents are in fiber form (SAF), obtained from Camelot, which are designated recognized as FIBERDRI® 1241 and FIBERDRI® 1161. Superabsorbents obtained from Technical Absorbents, Ltd. are designated OASIS® 101 and OASIS® 111. Another Example included in these types of superabsorbents is obtained from Chemtall Inc. and is designated FLOSORB® 60 Lady. Another Example included in these types of superabsorbents is obtained from Sumitomo Seika and is recognized as SA60N Type 2.

Binders may also be used in structures to help provide mechanical integrity and stabilization. Binders include fiberous, liquid or other binder means which may thermally activated. Preferred fibers for inclusion are those having a relative melting point such as polyolefin fibers. Lower melting point polymers provide the ability to bond nonwoven fabric together at fiber crossover points upon the application of heat. In addition, fibers having a lower melting polymer, like conjugate and biconstituent fibers are suitable for use as binders. Exemplary binder fibers include conjugate fibers of polyolefins, polyamides and polyesters like the sheath core conjugate fibers available from KoSa Inc. (Charlotte, N.C.) under the designation T-255 and T-256. A suitable liquid binder is KYMENE® 557LX available from Hercules Inc.

In this invention, a separate transfer delay is avoided in order to simplify manufacturing procedures and to reduce costs. A separate transfer delay layer is obviated by the use of an absorbent core or distribution layer having an inherent ability to delay the Z-directional transfer of fluid below the target area. This ability should predominate at low liquid loadings but should be overcome at higher liquid loadings, e.g. during gush flow conditions, due to pressure.

It should be noted that it may be possible to produce an absorbent core having a surface which is entirely less amenable to fluid acceptance than a conventional absorbent core. It is believed, however, that this would provide little incremental improvement over the instant invention, while increasing the possibility of leakage from the edges of the product.

There are a number of different ways of providing reduced fluid acceptance in the absorbent core immediately below target area. These include providing an absorbent core which: has a higher density below the target area then elsewhere, is rich in superabsorbent fibers in the area below the target area, is rich in slow superabsorbent in the area below the target area, has a soluble binder in the area below the target area, or one which has a hydrophobic treatment in the area below the target area, or a combination of these features. When an area is “rich in” a material, what is meant is that the area contains more of the material than is present in the material outside of the area. It should be noted that although treatments are contemplated for application to the absorbent core in the area below the target area, such treatment could be placed upon the bottom surface of the layer above the absorbent core, in this case the distribution layer.

Alternatively, a target area of increased density may be made by compressing the absorbent in only the target area. Alternatively, fibers of a smaller denier may be used in only the target area, and at a higher fiber count.

Superabsorbent fibers may be easily blended with the synthetic and natural fibers in a nonwoven process used to produce the absorbent core. If they are placed only, or primarily, in the target area, they will act to reduce the penetration of fluid into the core below by absorbing the fluid and expanding. This expansion will reduce void volume and further decrease fluid penetration into the core.

A related method of slowing fluid entry into the core is by using a mixture of superabsorbents in the absorbent core. Using a relatively fast absorbing superabsorbent (or mixture thereof) in the area outside the target area while using a relative slow absorbing superabsorbent (or mixture thereof) inside the target area will similarly discourage fluid penetration of the core in the target area and instead encourage X-Y plane fluid movement. The fluid can thus be detoured outside of the target area where it may be absorbed by the balance of the core. The relative speed of superabsorbents may be ascertained by the use of the absorption time index (ATI) test, given above. A greater ATI means that a superabsorbent is relatively slower than a superabsorbent having a lower ATI.

A soluble binder applied to the core or distribution layer below the target area will also function to delay fluid entry into the core by blocking pores. This is a direct physical method of restriction the penetration of fluid into the core. Such binders include certain latexes, polyvinyl alcohols, acrylate ester/acrylic or methylacrylic acid copolymers and blends. These polymers will block fluid entry, though not permanently, since one objective of the invention is to “delay” fluid entry, not stop it entirely. Once the binder has become soluble, most likely after the second or third insult, ease of fluid entry will approach that of areas of the absorbent core that are not below the target area.

A hydrophobic treatment may be applied to the core or distribution layer below the target area to delay fluid entry, or, conversely, a hydrophilic treatment may be applied to the absorbent core or distribution layer surface outside the target area. Suitable treatments include AHCOVEL® Base N-62 surfactant, which is a blend of about 50 weight percent sorbitan mono-oleate and about 50 weight percent hydrogenated ethoxylated castor oil at 100 percent solids supplied by ICI Chemicals, GLUCOPON® UP-220, an alkyl polyglycoside with a C8-10 chain at 60 percent solids, and many others known to those skilled in the art.

One example of a material according to this invention is one in which the lower surface of the distribution material is treated with a solution of AHCOVEL® surfactant in an amount from a positive amount to 0.1 weight percent. This may be applied by methods known in the art in an area outside the target area so that liquid movement in the Z-direction is enhanced. Another example of a material according to this invention is one having a concentration of superabsorbent in the target area of the absorbent core of 10 to 50 percent greater than the surrounding area. Still another example is one having a compressed area in the absorbent core, corresponding to the target area, which results in a density from 10 to 50 percent greater than the uncompressed absorbent core.

Personal care products made with the materials of this invention should more efficiently and completely use the absorbent core area since they will slow and spread out any liquid from an insult. Under high flow or “gush” conditions, however, liquid should pass through the layers above the core more easily, because of the pressure of the fluid, and be absorbed in the core.

As will be appreciated by those skilled in the art, changes and variations to the invention are considered to be within the ability of those skilled in the art. Examples of such changes are contained in the patents identified above, each of which is incorporated herein by reference in its entirety to the extent it is consistent with this specification. Such changes and variations are intended by the inventors to be within the scope of the invention.

What is claimed is:

1. A personal care absorbent article comprising a target area wherein fluid moves in a Z-direction at a slower rate in said target area than outside said target area.

2. The personal care article of claim 1 wherein an absorbent core has a higher density in the target area than the absorbent core outside the target area.

3. The personal care article of claim 1 wherein an absorbent core has more superabsorbent in the target area than outside the target area.

4. The personal care article of claim 1 wherein an absorbent core has a slower superabsorbent in the target area than outside the target area.

5. The personal care article of claim 1 wherein an absorbent core has a soluble binder in said target area.

6. The personal care article of claim 1 wherein an absorbent core has target area that is more hydrophobic than the absorbent core outside the target area.

7. The personal care article of claim 1 wherein a distribution layer has a soluble binder in said target area.

8. The personal care article of claim 1 wherein a distribution layer below the target area is more hydrophobic than the absorbent core outside the target area.

9. The personal care product of claim 1 chosen from the group consisting of feminine hygiene pads, diapers, training pants and incontinence products.

10. A feminine hygiene product comprising:
a baffle;
a body side liner having a target area, and;
an absorbent core disposed between said baffle and liner, comprising fluff;
wherein fluid moves in a Z -direction at a slower rate below said target area than outside said target area

11. A personal care article having an absorbent core with a higher density on the target area than the absorbent core has outside the target area.
